(19) United States
(12) Patent Application Publication
(10) Pub. No.: US 2026/0076735 A1
(43) Pub. Date: Mar. 19, 2026

(54) SYSTEMS AND METHODS FOR LACERATING HEART VALVE LEAFLETS

(71) Applicant: BOSTON SCIENTIFIC SCIMED, INC., MAPLE GROVE, MN (US)

(72) Inventors: Eric Michael Petersen, Maple Grove, MN (US); Philip Andrew Litecky, Forest Lake, MN (US); James M. Anderson, Corcoran, MN (US)

(73) Assignee: BOSTON SCIENTIFIC SCIMED, INC., MAPLE GROVE, MN (US)

(21) Appl. No.: 19/332,567

(22) Filed: Sep. 18, 2025

Related U.S. Application Data
(60) Provisional application No. 63/696,497, filed on Sep. 19, 2024.

(51) Int. Cl. A61B 18/14; A61B 18/00; A61M 25/00; A61M 25/01; A61M 25/06

(52) U.S. Cl. CPC A61B 18/1492; A61M 25/0071; A61M 25/0147; A61M 25/0662; A61B 2018/00184; A61B 2018/00214; A61B 2018/00273; A61B 2018/00369; A61B 2018/00577; A61B 2018/00601; A61M 2210/125

(57) ABSTRACT

A system for lacerating heart valve leaflets includes a coronary guidewire and a laceration device including an ablative cutting element. A system may include a first guidewire for insertion into a left ventricle and a guide catheter configured to translate along the first guidewire. The guide catheter may include a steering element configured to direct the coronary guidewire toward the coronary artery. The laceration device cooperates with the coronary guidewire to align the ablative cutting element with a nadir of a heart valve leaflet. A method of lacerating heart valve leaflets includes advancing the coronary guidewire into a coronary artery, translating the laceration device along the coronary guidewire to a position adjacent a heart valve leaflet, positioning the ablative cutting element in contact with a nadir of the heart valve leaflet, and lacerating the heart valve leaflet from the nadir to a free edge of the heart valve leaflet.

### CROSS-REFERENCE TO RELATED APPLICATIONS

This application claims the benefit of priority of U.S. Provisional Application No. 63/696,497 filed Sep. 19, 2024, the entire disclosure of which is hereby incorporated by reference.

### TECHNICAL FIELD

The disclosure relates generally to medical devices and more particularly to systems and methods pertaining to lacerating heart valve leaflets.

### BACKGROUND

A wide variety of intracorporeal medical devices have been developed for medical use including artificial heart valves for repair or replacement of diseased heart valves. Diseases and/or medical conditions that impact the cardiovascular system are prevalent throughout the world. Some relatively common medical conditions may include or be the result of inefficiency, ineffectiveness, or complete failure of one or more of the valves within the heart. Treatment of defective heart valves poses other challenges in that the treatment often requires the repair or outright replacement of the defective valve.

In some patients, initial implantation of a replacement heart valve implant and/or a subsequent treatment involving a second replacement heart valve implant disposed therein must ensure that the ostia of the coronary arteries do not become partially or completely obstructed, which may be a life-threatening complication. Irregular anatomy shapes and/or configurations may render this possibility more likely.

When treating the coronary arteries, the coronary ostia are accessed from within the aortic sinus. A leaflet splitting procedure known as “basilica” has been developed that modifies the leaflets of the native heart valve and/or the initial replacement heart valve implant prior to implantation of the initial replacement heart valve implant and/or the second replacement heart valve implant, respectively, to improve access to the coronary ostia. Proper location of the initial penetration point for the basilica procedure relative to the coronary ostia is necessary to ensure the coronary ostia remain unobstructed after the procedure. There is an ongoing need to provide alternative medical devices and/or systems as well as alternative methods for manufacturing and using medical devices and/or systems.

### SUMMARY

In a first example, a system for lacerating heart valve leaflets may comprise a coronary guidewire having a distal end configured for insertion into a coronary artery, and a laceration device configured to translate along the coronary guidewire to a position adjacent a heart valve leaflet disposed proximate the coronary artery, wherein the laceration device comprises an ablative cutting element. The laceration device may cooperate with the coronary guidewire to align the ablative cutting element with a nadir of the heart valve leaflet.

In addition, or alternatively, to any example disclosed herein, the distal end of the coronary guidewire comprises an expandable structure configured to anchor the coronary guidewire within the coronary artery in an expanded configuration.

In addition, or alternatively, to any example disclosed herein, the expandable structure is configured to permit blood to flow through the coronary artery past the expandable structure in the expanded configuration.

In addition, or alternatively, to any example disclosed herein, the laceration device is slidably coupled to the coronary guidewire such that the laceration device is offset laterally from the coronary guidewire.

In addition, or alternatively, to any example disclosed herein, the laceration device comprises an expandable member disposed at a distal end of the laceration device, wherein the ablative cutting element is disposed on the expandable member.

In addition, or alternatively, to any example disclosed herein, the laceration device comprises a device catheter comprising a guidewire lumen and a working lumen separate from the guidewire lumen, wherein the coronary guidewire is configured to extend through the guidewire lumen and the ablative cutting element is configured to extend through the working lumen.

In addition, or alternatively, to any example disclosed herein, the device catheter is bifurcated proximate its distal end such that the guidewire lumen and the working lumen diverge from each other.

In addition, or alternatively, to any example disclosed herein, the device catheter is configured such that with the coronary guidewire disposed within the coronary artery, the working lumen extends toward the nadir of the heart valve leaflet.

In addition, or alternatively, to any example disclosed herein, and in another example, a system for lacerating heart valve leaflets may comprise a first guidewire configured for insertion into a left ventricle of a heart, a guide catheter configured to translate along the first guidewire to a position adjacent a heart valve leaflet disposed proximate the coronary artery, a laceration device slidably disposed within the guide catheter, the laceration device comprising an ablative cutting element, and a coronary guidewire having a distal end configured for insertion into a coronary artery. The guide catheter may comprise a steering element configured to direct the coronary guidewire toward the coronary artery. The laceration device may cooperate with the coronary guidewire to align the ablative cutting element with a nadir of the heart valve leaflet.

In addition, or alternatively, to any example disclosed herein, the steering element is a pull wire configured to deflect a distal end of the guide catheter toward the coronary artery.

In addition, or alternatively, to any example disclosed herein, the steering element is an expandable frame configured to shift from a delivery configuration to a deployed configuration to deflect a distal end of the guide catheter toward the coronary artery.

In addition, or alternatively, to any example disclosed herein, the system may comprise a delivery catheter configured to constrain the expandable frame in the delivery configuration during translation of the guide catheter along the first guidewire, wherein proximal movement of the delivery catheter relative to the guide catheter permits the expandable frame to shift to the deployed configuration.

In addition, or alternatively, to any example disclosed herein, the expandable frame is fixedly attached to the guide catheter.

In addition, or alternatively, to any example disclosed herein, the expandable frame is slidably coupled to the first guidewire.

In addition, or alternatively, to any example disclosed herein, and in another example, a method of lacerating heart valve leaflets may comprise: advancing a coronary guidewire into a coronary artery; translating a laceration device along the coronary guidewire to a position adjacent a heart valve leaflet disposed proximate the coronary artery, wherein the laceration device comprises an ablative cutting element; positioning the ablative cutting element in contact with a nadir of the heart valve leaflet; and lacerating the heart valve leaflet from the nadir of the heart valve leaflet to a free edge of the heart valve leaflet.

In addition, or alternatively, to any example disclosed herein, the method may comprise: before translating the laceration device along the coronary guidewire, anchoring the coronary guidewire within the coronary artery.

In addition, or alternatively, to any example disclosed herein, positioning the ablative cutting element in contact with the nadir of the heart valve leaflet comprises shifting an expandable member from a collapsed configuration to an expanded configuration.

In addition, or alternatively, to any example disclosed herein, the expandable member is disposed at a distal end of a device catheter and the coronary guidewire is disposed within a guidewire lumen of the device catheter, wherein the coronary guidewire exits the guidewire lumen proximal of the expandable member.

In addition, or alternatively, to any example disclosed herein, the laceration device comprises a device catheter comprising a guidewire lumen and a working lumen separate from the guidewire lumen, wherein the coronary guidewire is configured to extend through the guidewire lumen and the ablative cutting element is configured to extend through the working lumen.

In addition, or alternatively, to any example disclosed herein, positioning the ablative cutting element in contact with the nadir of the heart valve leaflet comprises: orienting the device catheter, with the coronary guidewire disposed within the coronary artery, such that the working lumen opens toward the nadir of the heart valve leaflet; and advancing the ablative cutting element out of the working lumen into contact with the nadir of the heart valve leaflet.

The above summary of some embodiments, aspects, and/or examples is not intended to describe each disclosed embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIG. 1 is a top view schematically illustrating selected aspects of a heart valve;

FIG. 2 is a partial cutaway view schematically illustrating selected aspects of a heart valve;

FIG. 3 illustrates a coronary guidewire inserted into the left coronary artery;

FIG. 4 illustrates selected aspects of a system for lacerating heart valve leaflets;

FIGS. 5-7 illustrate selected aspects of a system for and a method of lacerating heart valve leaflets;

FIG. 8 illustrates selected aspects of a system for lacerating heart valve leaflets;

FIG. 9 illustrates selected aspects of a system for lacerating heart valve leaflets;

FIGS. 10-11 illustrate selected aspects of a system for lacerating heart valve leaflets;

FIG. 12 illustrates selected aspects of a system for lacerating heart valve leaflets;

FIG. 13 illustrates selected aspects of a system for lacerating heart valve leaflets;

FIGS. 14-15 illustrate selected aspects of a system for lacerating heart valve leaflets; and

FIGS. 16-21 illustrate selected aspects of a coronary guidewire according to the disclosure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale and/or which may include changes of scale therein. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

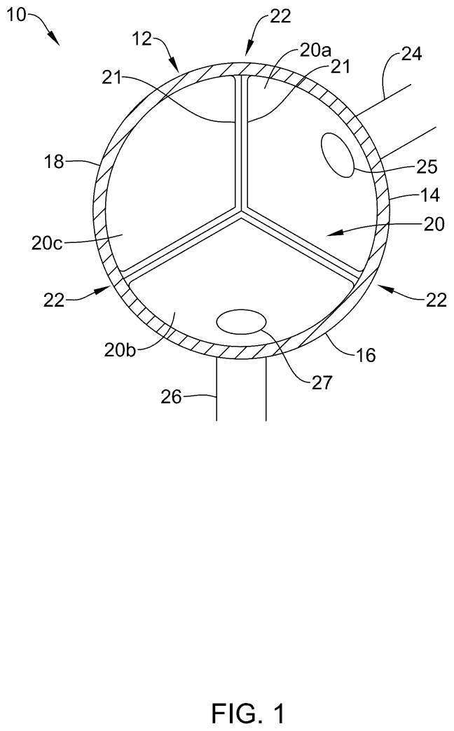

FIG. 1 illustrates selected aspects of a native heart valve, such as an aortic valve 10, of a patient's heart. FIG. 1 illustrates a partial cross-sectional taken through the ascending aorta 12 just downstream from the aortic valve 10. The aortic valve 10 may comprise a left coronary cusp 14, a right coronary cusp 16, and a non-coronary cusp 18. The aortic valve 10 comprises a plurality of heart valve leaflets 20, and one heart valve leaflet is associated with each cusp. Circumferentially adjacent heart valve leaflets of the plurality of heart valve leaflets 20 come together at and/or share commissures 22. For the purpose of this disclosure, the plurality of heart valve leaflets 20 comprises a left heart valve leaflet 20a, a right heart valve leaflet 20b, and a non-coronary heart valve leaflet 20c. These designations are made arbitrarily to aid in understanding the disclosure.

The patient's heart comprises a left coronary artery 24 extending from the left coronary cusp 14 downstream of the left heart valve leaflet 20a, and a right coronary artery 26 extending from the right coronary cusp 16 downstream of the right heart valve leaflet 20b. The left coronary artery 24 opens to the ascending aorta 12 and/or the left coronary cusp 14 at a left coronary ostium 25. The right coronary artery 26 opens to the ascending aorta 12 and/or the right coronary cusp 16 at a right coronary ostium 27. The plurality of heart valve leaflets 20 shift between an open position and a closed position (as seen in FIG. 1) in rhythm with blood flow from the left ventricle of the patient's heart. In the closed position, free edges 21 of the plurality of heart valve leaflets 20 may come together to substantially restrict blood flow through the aortic valve 10. In the open position, the free edges 21 of the plurality of heart valve leaflets 20 may move apart from each other to permit blood flow through the aortic valve 10.

While FIG. 1 illustrates the aortic valve 10 in an ideal configuration (e.g., features thereof are equally sized and/or spaced, well aligned, etc.), the aortic valve of real-world patients may have anatomical structure and/or arrangement that varies from the ideal configuration. The preferred placement of the initial puncture point for the basilica procedure is at the nadir of the heart valve leaflet in front of the associated coronary artery with an axial laceration to the free edge of the heart valve leaflet. Proper location of the initial puncture point may be difficult due to variations in patient anatomy, which may result in the initial puncture point and subsequent laceration being misaligned with the associated coronary artery. Discussed herein are systems, devices, and methods which may improve location and/or alignment of a laceration in a heart valve leaflet with respect to its associated coronary artery.

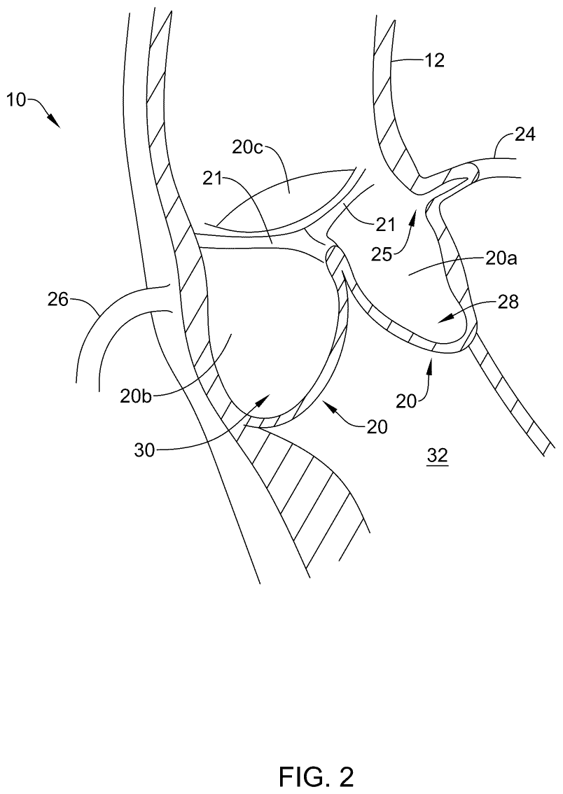

FIG. 2 is a partial cutaway view of the aortic valve 10, wherein a portion of the ascending aorta 12 has been cutaway to show the plurality of heart valve leaflets 20, including the left heart valve leaflet 20a, the right heart valve leaflet 20b, and the non-coronary heart valve leaflet 20c. The left coronary artery 24 is disposed adjacent to the left heart valve leaflet 20a and the right coronary artery 26 is disposed adjacent to the right heart valve leaflet 20b. The free edges 21 of the plurality of heart valve leaflets 20 may come together to substantially restrict blood flow through the aortic valve 10 in the closed position, as shown. FIG. 2 also identifies a nadir 28 of the left heart valve leaflet 20a and a nadir 30 of the right heart valve leaflet 20b. Below and/or upstream of the aortic valve 10 and/or the plurality of heart valve leaflets 20 lies the left ventricle 32. Above and/or downstream of the aortic valve 10 and/or the plurality of heart valve leaflets 20 lies the ascending aorta 12. In FIG. 2, the left coronary ostium 25 is visible while the right coronary ostium 27 is not visible.

In at least some embodiments associated with the disclosure, the left coronary artery 24 and/or the right coronary artery 26 may be used to locate the preferred placement of the initial puncture point for the basilica procedure for the respective heart valve leaflet. For the purpose of the disclosure, discussion will be directed toward the left coronary artery 24. However, it shall be understood that the same devices, procedures, and/or methods may be used with the right coronary artery 26, either independently of the left coronary artery 24 or in association with the left coronary artery 24 (e.g., both the left coronary artery 24 and the right coronary artery 26 may be accessed, used, etc.).

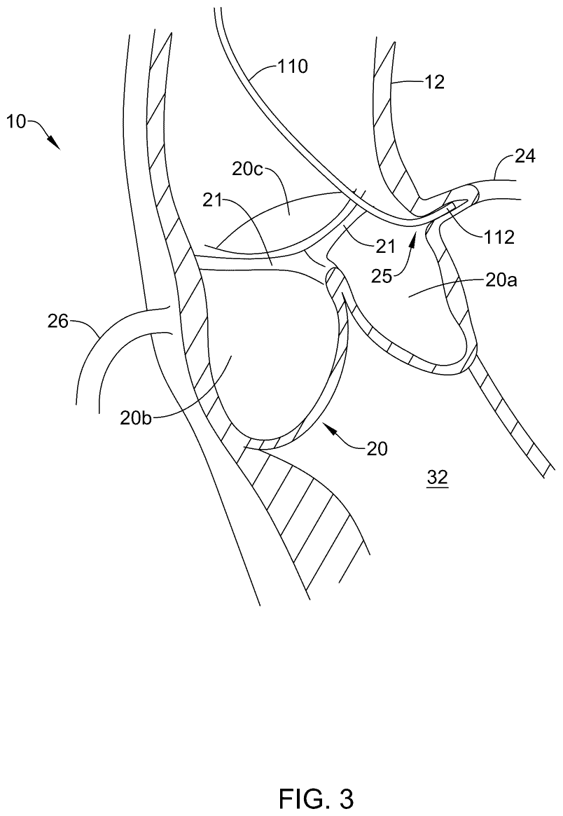

FIG. 3 illustrates a coronary guidewire 110 inserted into the left coronary ostium 25 and/or the left coronary artery 24. The coronary guidewire 110 may have a distal end 112 configured for insertion into a coronary artery (e.g., the left coronary artery 24 and/or the right coronary artery 26). In some embodiments, the coronary guidewire 110 may be advanced percutaneously to the left coronary ostium 25 and/or the left coronary artery 24 alone or independently of any other device. In some embodiments, the coronary guidewire 110 may be advanced percutaneously to the left coronary ostium 25 and/or the left coronary artery 24 in conjunction with another device. In some embodiments, the coronary guidewire 110 may be advanced percutaneously to the left coronary ostium 25 and/or the left coronary artery 24 within a delivery catheter. In some embodiments, the delivery catheter may be tracked over a guidewire or other device. Other configurations are also contemplated.

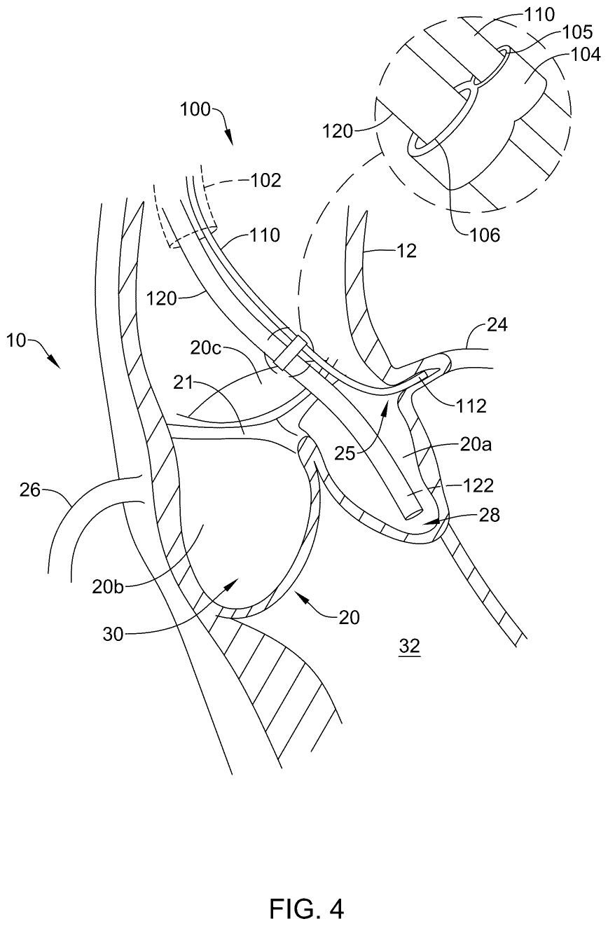

FIG. 4 illustrates selected aspects of a system 100 for lacerating heart valve leaflets. In some embodiments, the system 100 may comprise the coronary guidewire 110. As shown in and/or described with respect to FIG. 3, the coronary guidewire 110 may have a distal end 112 configured for insertion into a coronary artery (e.g., the left coronary artery 24 and/or the right coronary artery 26). In some embodiments, the system 100 may comprise a laceration device 120 configured to translate along the coronary guidewire 110 to a position adjacent a heart valve leaflet (e.g., the left heart valve leaflet 20a, the right heart valve leaflet 20b, etc.) disposed proximate the coronary artery (e.g., the left coronary artery 24 and/or the right coronary artery 26). In some embodiments, the laceration device 120 may comprise a tubular sheath. In some embodiments, the laceration device 120 may comprise an ablative cutting element 122 (not visible in FIG. 4). In some embodiments, the ablative cutting element 122 may be a conductive wire (e.g., a guidewire etc.) slidably disposed within the laceration device 120 and/or the tubular sheath. In some embodiments, the ablative cutting element 122 may be configured to be electrically coupled to a controller and/or an energy source, such as but not limited to a radiofrequency (RF) energy source, an RF generator, etc. In some embodiments, the ablative cutting element 122 may be formed from and/or may be enhanced with a highly radiopaque material and/or highly radiopaque features, elements, markers, etc. In some embodiments, the highly radiopaque material and/or highly radiopaque features, elements, markers, etc. may enhance device navigation, and/or facilitate and/or improve anatomical alignment. Other configurations are also contemplated.

In some embodiments, the laceration device 120 may be slidably coupled to the coronary guidewire 110 such that the laceration device 120 is offset laterally from the coronary guidewire 110. In some embodiments, the laceration device 120 may be slidably coupled to the coronary guidewire 110 such that the laceration device 120 is disposed non-concentric with the coronary guidewire 110. In some embodiments, the laceration device 120 may be slidably coupled to the coronary guidewire 110 such that the laceration device 120 is disposed eccentrically with respect to the coronary guidewire 110. In some embodiments, the laceration device 120 may be slidably coupled to the coronary guidewire 110 such that the laceration device 120 is disposed alongside of the coronary guidewire 110. In some embodiments, the laceration device 120 may be slidably coupled to the coronary guidewire 110 such that the coronary guidewire 110 is disposed outside of and/or exterior to the laceration device 120. In some embodiments, the laceration device 120 may be slidably coupled to the coronary guidewire 110 such that the coronary guidewire 110 is disposed completely outside of and/or completely exterior to the laceration device 120.

In some embodiments, the laceration device 120 may be slidably coupled to the coronary guidewire 110 by a sled 104. In some embodiments, the sled 104 may comprise a first lumen 105 and a second lumen 106 disposed side-by-side and/or laterally offset from each other. In some embodiments, the first lumen 105 and the second lumen 106 may be oriented parallel to each other. In some embodiments, the first lumen 105 may be sized and configured to slidably receive the coronary guidewire 110. In some embodiments, the second lumen 106 may be sized and configured to receive the laceration device 120. In some embodiments, the sled 104 may be non-slidably coupled to the laceration device 120. In some embodiments, the sled 104 may be fixedly attached to the laceration device 120. In some embodiments, the second lumen 106 may be sized and configured to slidably receive the laceration device 120. In some alternative embodiments, the laceration device 120 may be slidably coupled to the coronary guidewire 110 by a plurality of sleds. Other configurations are also contemplated.

In some embodiments, the coronary guidewire 110 and/or the laceration device 120 may optionally be slidably disposed within and/or may optionally be advanced toward the aortic valve 10 within a delivery catheter 102 (shown in dashed lines in FIG. 4). In some embodiments, the delivery catheter 102 may slidably couple the laceration device 120 to the coronary guidewire 110. In some embodiments, the system 100 may comprise the delivery catheter 102 or the sled 104 (or the plurality of sleds). In some embodiments, the system 100 may comprise the delivery catheter 102 and the sled 104. In some embodiments, the system 100 may comprise the delivery catheter 102 and the plurality of sleds.

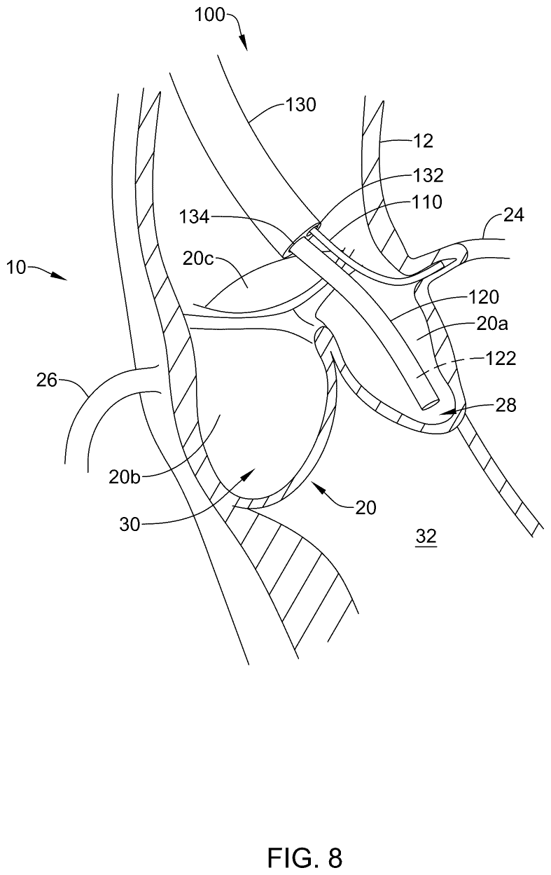

In some embodiments, the laceration device 120 may comprise a device catheter 130 (e.g., FIGS. 8-11). In some embodiments, the device catheter 130 may be used in the alternative to the sled 104. In some embodiments, the device catheter 130 may be used in addition to the delivery catheter 102. Additional details regarding the device catheter 130 are provided below.

In some embodiments, the laceration device 120 may be configured to cooperate with the coronary guidewire 110 to align the laceration device 120 and/or the ablative cutting element 122 with the coronary guidewire 110, the coronary artery (e.g., the left coronary artery 24, the right coronary artery 26) associated with a heart valve leaflet (e.g., the left heart valve leaflet 20a, the right heart valve leaflet 20b) being lacerated, and/or the nadir (e.g., the nadir 28, the nadir 30) of the heart valve leaflet (e.g., the left heart valve leaflet 20a, the right heart valve leaflet 20b) being lacerated. In some embodiments, the laceration device 120 may be configured to cooperate with the coronary guidewire 110 to align the laceration device 120 and/or the ablative cutting element 122 with the nadir (e.g., the nadir 28, the nadir 30) of the heart valve leaflet (e.g., the left heart valve leaflet 20a, the right heart valve leaflet 20b). Other configurations are also contemplated. In some embodiments, the laceration device 120 and/or the device catheter 130 may be aligned with the coronary guidewire 110 under fluoroscopy (or another suitable visualization means) using one or more views such that a distal end of the laceration device 120 and/or the device catheter 130 may be directed toward and/or to the nadir (e.g., the nadir 28, the nadir 30) of the heart valve leaflet (e.g., the left heart valve leaflet 20a, the right heart valve leaflet 20b). In some embodiments, the laceration device 120 may comprise one or more radiopaque markers configured to facilitate aligning the laceration device 120 and/or the ablative cutting element 122 with the coronary guidewire 110 and/or the nadir (e.g., the nadir 28, the nadir 30) of the heart valve leaflet (e.g., the left heart valve leaflet 20a, the right heart valve leaflet 20b).

FIGS. 3-7 illustrate selected aspects of the system 100 for lacerating heart valve leaflets and a method of lacerating heart valve leaflets. In some embodiments, the method of lacerating heart valve leaflets (e.g., the left heart valve leaflet 20a and/or the right heart valve leaflet 20b) may comprise advancing the coronary guidewire 110 into the coronary artery (e.g., the left coronary artery 24 and/or the right coronary artery 26). In some embodiments, the method of lacerating heart valve leaflets (e.g., the left heart valve leaflet 20a and/or the right heart valve leaflet 20b) may comprise translating the laceration device 120 and/or the device catheter 130 along the coronary guidewire 110 to a position adjacent a heart valve leaflet (e.g., the left heart valve leaflet 20a and/or the right heart valve leaflet 20b) disposed proximate the coronary artery (e.g., the left coronary artery 24 and/or the right coronary artery 26). In some embodiments, before translating the laceration device 120 and/or the device catheter 130 along the coronary guidewire 110, the method of lacerating heart valve leaflets (e.g., the left heart valve leaflet 20a and/or the right heart valve leaflet 20b) may comprise anchoring the coronary guidewire 110 within the coronary artery (e.g., the left coronary artery 24 and/or the right coronary artery 26), as discussed herein.

In some embodiments, as seen in FIG. 4, the laceration device 120 and/or the device catheter 130 (e.g., FIG. 8) may be configured to position a distal end of the laceration device 120 and/or the device catheter 130 adjacent to the nadir (e.g., the nadir 28, the nadir 30) of the heart valve leaflet (e.g., the left heart valve leaflet 20a, the right heart valve leaflet 20b). In some embodiments, the method of lacerating heart valve leaflets may comprise positioning the ablative cutting element 122 in contact with the nadir (e.g., the nadir 28, the nadir 30) of the heart valve leaflet (e.g., the left heart valve leaflet 20a, the right heart valve leaflet 20b). In some embodiments, positioning the ablative cutting element 122 in contact with the nadir (e.g., the nadir 28, the nadir 30) of the heart valve leaflet (e.g., the left heart valve leaflet 20a, the right heart valve leaflet 20b) comprises orienting the device catheter 130, with the coronary guidewire 110 disposed within the coronary artery (e.g., the left coronary artery 24 and/or the right coronary artery 26), such that a working lumen 134 (FIG. 8) opens toward the nadir (e.g., the nadir 28, the nadir 30) of the heart valve leaflet (e.g., the left heart valve leaflet 20a, the right heart valve leaflet 20b).

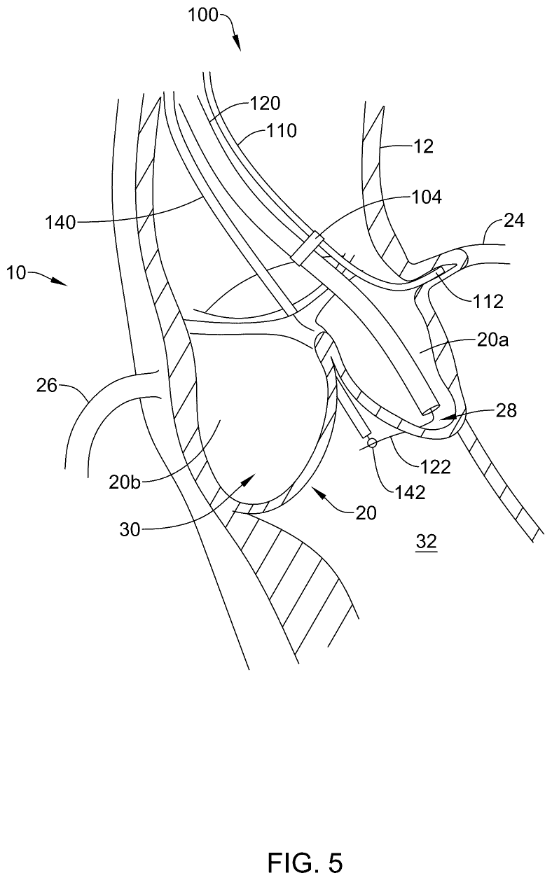

In some embodiments, positioning the ablative cutting element 122 in contact with the nadir (e.g., the nadir 28, the nadir 30) of the heart valve leaflet (e.g., the left heart valve leaflet 20a, the right heart valve leaflet 20b) comprises advancing the ablative cutting element 122 out of the working lumen 134 and in contact with the nadir (e.g., the nadir 28, the nadir 30) of the heart valve leaflet (e.g., the left heart valve leaflet 20a, the right heart valve leaflet 20b). In at least some embodiments, positioning the ablative cutting element 122 in contact with the nadir (e.g., the nadir 28, the nadir 30) of the heart valve leaflet (e.g., the left heart valve leaflet 20a, the right heart valve leaflet 20b) comprises extending the ablative cutting element 122 through the nadir (e.g., the nadir 28, the nadir 30) of the heart valve leaflet (e.g., the left heart valve leaflet 20a, the right heart valve leaflet 20b), as seen in FIG. 5. As discussed herein, the ablative cutting element 122 may be and/or may comprise a conductive wire. In some embodiments, positioning the ablative cutting element 122 in contact with the nadir (e.g., the nadir 28, the nadir 30) of the heart valve leaflet (e.g., the left heart valve leaflet 20a, the right heart valve leaflet 20b) comprises extending the conductive wire through the nadir (e.g., the nadir 28, the nadir 30) of the heart valve leaflet (e.g., the left heart valve leaflet 20a, the right heart valve leaflet 20b). In some embodiments, the ablative cutting element 122 and/or the conductive wire may be configured to puncture the nadir (e.g., the nadir 28, the nadir 30) of the heart valve leaflet (e.g., the left heart valve leaflet 20a, the right heart valve leaflet 20b).

In some embodiments, the system 100 for lacerating heart valve leaflets may comprise a second device catheter 140. The second device catheter 140 may be configured to be advanced percutaneously through the plurality of heart valve leaflets 20 such that a distal end of the second device catheter 140 is disposed upstream of and/or on an upstream side of the plurality of heart valve leaflets 20, as seen in FIG. 5. Accordingly, the method of lacerating heart valve leaflets may comprise advancing the second device catheter 140 through the plurality of heart valve leaflets 20 and/or positioning a distal end of the second device catheter 140 upstream of and/or on an upstream side of the plurality of heart valve leaflets 20.

The second device catheter 140 may comprise a snare 142. In some embodiments, the snare 142 may extend from the second device catheter 140. In some embodiments, the snare 142 may be slidably disposed within the second device catheter 140 and be configured to be extended from the second device catheter 140. In some embodiments, the snare 142 may be configured to capture and/or grasp the ablative cutting element 122 and/or the conductive wire. In some embodiments, the snare 142 may comprise a closed loop. In some embodiments, the snare 142 may comprise a cinch loop. In some embodiments, the snare 142 may comprise an actuatable grasping element, such as a clip (e.g., an alligator clip). In some embodiments, capturing and/or grasping the ablative cutting element 122 and/or the conductive wire may comprise closing the snare 142 around and/or onto the ablative cutting element 122 and/or the conductive wire. The method of lacerating heart valve leaflets may comprise capturing and/or grasping the ablative cutting element 122 and/or the conductive wire with the snare 142.

In at least some embodiments, the snare 142 may be configured to pull a distal end portion of the ablative cutting element 122 and/or the conductive wire into the second device catheter 140. In some embodiments, the method of lacerating heart valve leaflets may comprise, after capturing and/or grasping the ablative cutting element 122 and/or the conductive wire with the snare 142, pulling the distal end portion of the ablative cutting element 122 and/or the conductive wire into the second device catheter 140.

In some embodiments, the snare 142 may be formed from a metallic material (e.g., nickel-titanium alloy, stainless steel and/or stainless-steel alloy, etc.), a polymeric material (e.g., nylon, aramid, PET, graphene, etc.), or a composite material. In some embodiments, the snare 142 may be formed from a high-tensile fiber or filament. In some embodiments, non-metallic materials may be doped with a radiopaque media (e.g., iodine, tungsten, platinum-iridium alloy, etc.). Other configurations are also contemplated. Some suitable but non-limiting materials for the snare 142, etc., including but not limited to polymeric materials, metallic materials, and/or composite materials, are described below.

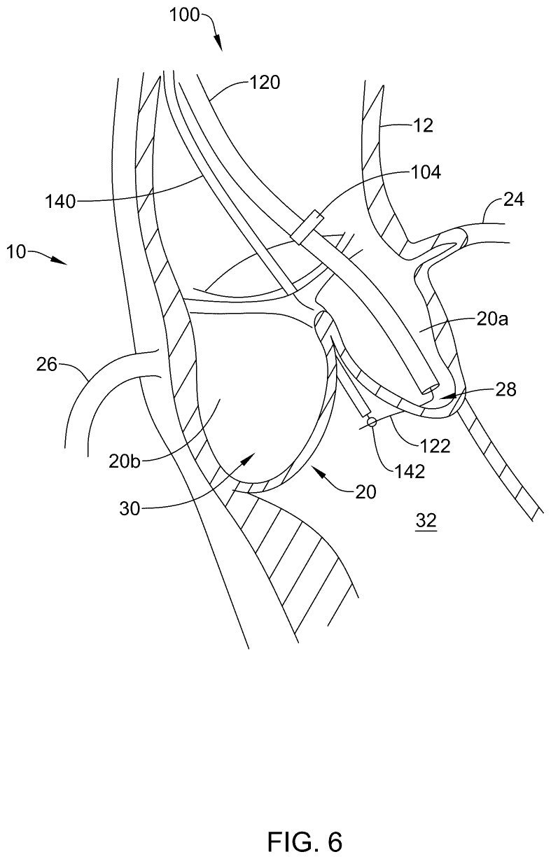

In some embodiments, the method of lacerating heart valve leaflets may comprise withdrawing and/or removing the coronary guidewire 110 from the coronary artery (e.g., the left coronary artery 24 and/or the right coronary artery 26), as seen in FIG. 6. In some embodiments, withdrawing and/or removing the coronary guidewire 110 from the coronary artery (e.g., the left coronary artery 24 and/or the right coronary artery 26) may comprise withdrawing and/or removing the coronary guidewire 110 from the patient and/or the patient's vasculature. In some embodiments, withdrawing and/or removing the coronary guidewire 110 from the coronary artery (e.g., the left coronary artery 24 and/or the right coronary artery 26) may comprise withdrawing the coronary guidewire 110 into the delivery catheter 102 (e.g., FIG. 4). In some embodiments, withdrawing and/or removing the coronary guidewire 110 from the coronary artery (e.g., the left coronary artery 24 and/or the right coronary artery 26) may comprise withdrawing the coronary guidewire 110 from the sled 104 (e.g., FIG. 4). In some alternative embodiments, the coronary guidewire 110 may be left in place and/or left disposed within the coronary artery (e.g., the left coronary artery 24 and/or the right coronary artery 26) for the duration of the procedure. Other configurations are also contemplated.

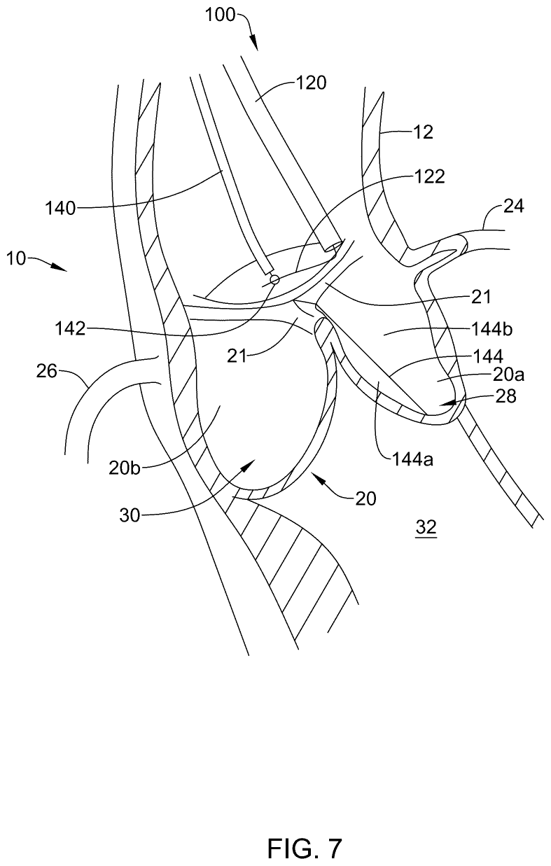

In some embodiments, the method of lacerating heart valve leaflets may comprise lacerating the heart valve leaflet (e.g., the left heart valve leaflet 20a, the right heart valve leaflet 20b) from the nadir (e.g., refs. 28, 30) of the heart valve leaflet (e.g., the left heart valve leaflet 20a, the right heart valve leaflet 20b) to a free edge (e.g., ref. 21) of the heart valve leaflet (e.g., the left heart valve leaflet 20a, the right heart valve leaflet 20b), as seen in FIG. 7 (ref. 144). In some embodiments, lacerating the heart valve leaflet (e.g., the left heart valve leaflet 20a, the right heart valve leaflet 20b) may comprise translating the laceration device 120 and/or the device catheter 130 proximally along with and/or concurrently with the second device catheter 140 to lacerate the heart valve leaflet (e.g., the left heart valve leaflet 20a, the right heart valve leaflet 20b) axially and/or in a downstream direction aligned with the ostium (e.g., the left coronary ostium 25, the right coronary ostium 27) of the coronary artery (e.g., the left coronary artery 24, the right coronary artery 26) such that the heart valve leaflet (e.g., the left heart valve leaflet 20a, the right heart valve leaflet 20b) is cut into two adjacent portions (refs. 144a, 144b).

In at least some embodiments, prior to lacerating the heart valve leaflet (e.g., the left heart valve leaflet 20a, the right heart valve leaflet 20b), the method may comprise energizing the ablative cutting element 122 and/or the conductive wire. In some embodiments, energizing the ablative cutting element 122 and/or the conductive wire may comprise applying RF energy to the ablative cutting element 122 and/or the conductive wire. Other configurations are also contemplated.

Some suitable but non-limiting materials for the delivery catheter 102, the sled 104, the coronary guidewire 110, the laceration device 120, the device catheter 130, the second device catheter 140, the snare 142, etc., including but not limited to polymeric materials, metallic materials, and/or composite materials, are described below.

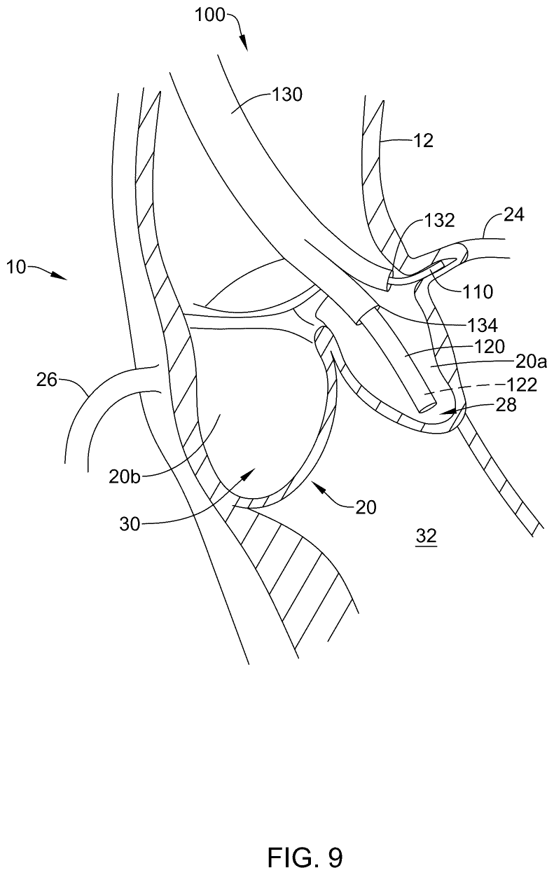

In some embodiments, the laceration device 120 may comprise a device catheter 130. In some embodiments, the device catheter 130 may be used in the alternative to the sled 104. In some embodiments, the device catheter 130 may be used in addition to the delivery catheter 102. In some embodiments, the device catheter 130 may comprise a guidewire lumen 132 and a working lumen 134 separate from the guidewire lumen 132, as seen in FIGS. 8-9. In at least some embodiments, the guidewire lumen 132 and the working lumen 134 may be completely separated and/or independent from each other (e.g., the guidewire lumen 132 and the working lumen 134 do not coexist within a common lumen). Other configurations are also contemplated. For example, in some alternative embodiments, a portion of the guidewire lumen 132 and a portion of the working lumen 134 may share and/or coexist within a common lumen or a portion of a lumen. However, such a configuration may not be preferred due to the risk of entanglement of the coronary guidewire 110 with the laceration device 120, which may increase operational forces, or cause other complications. In some embodiments, the coronary guidewire 110 may be sized and configured to extend through the guidewire lumen 132. In some embodiments, the laceration device 120, the ablative cutting element 122, and/or the conductive wire may be sized and configured to extend through the working lumen 134.

In some embodiments, the device catheter 130 may comprise a multi-lumen tubular member, as seen in FIG. 8 for example. In some embodiments, the device catheter 130 may comprise two or more nested tubular members (e.g., one or more tubular members may be disposed within an outer tubular member). In some embodiments, the device catheter 130 may comprise a multi-lumen tubular member that is bifurcated proximate its distal end such that the guidewire lumen 132 and the working lumen 134 may diverge from each other, as seen in FIG. 9 for example. In some embodiments, the device catheter 130 may be configured such that with the coronary guidewire 110 disposed within the coronary artery (e.g., the left coronary artery 24, the right coronary artery 26), the working lumen 134 may extend away from the coronary artery (e.g., the left coronary artery 24, the right coronary artery 26) toward the nadir (e.g., refs. 28, 30) of the heart valve leaflet (e.g., the left heart valve leaflet 20a, the right heart valve leaflet 20b). In some embodiments, after the coronary guidewire 110 is advanced into the coronary artery (e.g., the left coronary artery 24, the right coronary artery 26), the device catheter 130 may be advanced and/or translated along the coronary guidewire 110. The guidewire lumen 132 may follow the coronary guidewire 110 and/or deflect toward the coronary artery (e.g., the left coronary artery 24, the right coronary artery 26) while the working lumen 134 may extend and/or advance upstream toward the nadir (e.g., refs. 28, 30) of the heart valve leaflet (e.g., the left heart valve leaflet 20a, the right heart valve leaflet 20b). The laceration device 120 may be extended from the working lumen 134 of the device catheter 130 toward the nadir (e.g., refs. 28, 30) of the heart valve leaflet (e.g., the left heart valve leaflet 20a, the right heart valve leaflet 20b).

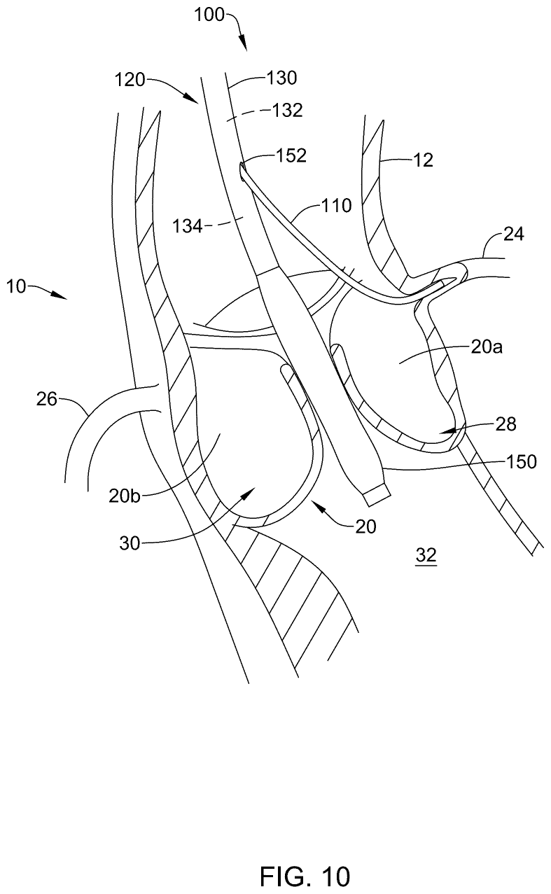

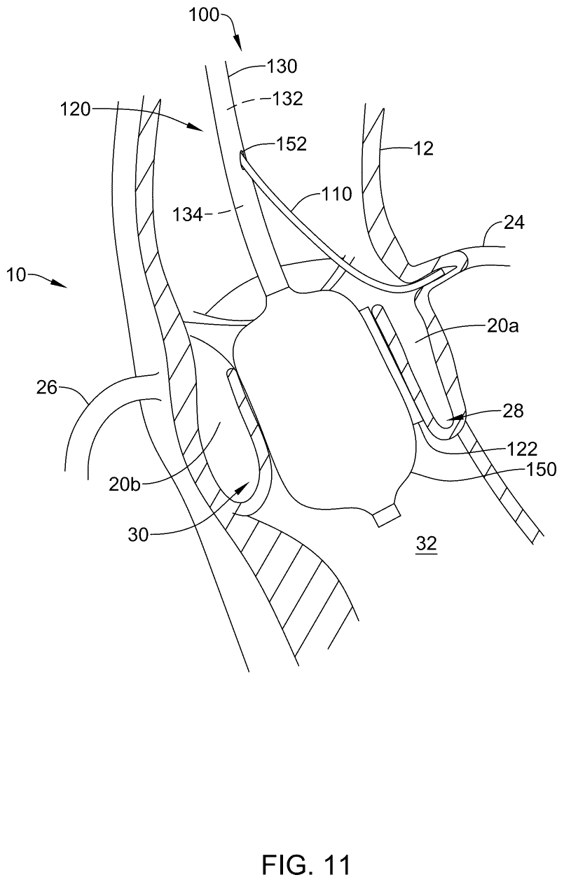

In some embodiments, the laceration device 120 may comprise an expandable member 150 disposed at a distal end of the device catheter 130, as seen in FIGS. 10-11. In some embodiments, the expandable member 150 may be an expandable framework fixedly attached to the device catheter 130. In some embodiments, the expandable member 150 may be a stent or an endoprosthesis fixedly attached to the device catheter 130. In some embodiments, the expandable member 150 may comprise an inflatable balloon, wherein the working lumen 134 of the device catheter 130 functions as an inflation lumen. In at least some embodiments, the coronary guidewire 110 may be configured to be disposed within the guidewire lumen 132 of the device catheter 130.

In some embodiments, the device catheter 130 may comprise a guidewire port 152 disposed proximal of the distal end. The guidewire port 152 may be in fluid communication with the guidewire lumen 132. In at least some embodiments, the guidewire port 152 may be disposed proximal of the expandable member 150. In some embodiments, the guidewire port 152 may open laterally relative to a central longitudinal axis of the device catheter 130 and/or the guidewire lumen 132. In some embodiments, the coronary guidewire 110 may exit the guidewire lumen 132 and/or the guidewire port 152 proximal of the expandable member 150.

In some embodiments, after advancing the coronary guidewire 110 into the coronary artery (e.g., the left coronary artery 24, the right coronary artery 26), the laceration device 120 and/or the device catheter 130 may be advanced over and/or translated along the coronary guidewire 110 to a position adjacent the heart valve leaflet (e.g., the left heart valve leaflet 20a, the right heart valve leaflet 20b) with the expandable member 150 disposed in a collapsed configuration, as seen in FIG. 10. The expandable member 150 may be advanced into the aortic valve 10 and/or within the plurality of heart valve leaflets 20 in the collapsed configuration.

In some embodiments, the ablative cutting element 122 may be disposed on the expandable member 150, as seen in FIG. 11. In some embodiments, the ablative cutting element 122 may be disposed on an outer surface of the expandable member 150. In some embodiments, the ablative cutting element 122 may face radially outward from the expandable member 150. In some alternative embodiments, a first portion of the ablative cutting element 122 may be embedded within the expandable member 150 and a second portion of the ablative cutting element 122 may extend radially outward from the expandable member 150. Other configurations are also contemplated.

After advancing the expandable member 150 into the aortic valve 10 and/or within the plurality of heart valve leaflets 20 in the collapsed configuration, the expandable member 150 and/or the ablative cutting element 122 may be aligned with and/or oriented relative to the coronary guidewire 110 and/or the coronary artery (e.g., the left coronary artery 24, the right coronary artery 26) to position the ablative cutting element 122 against the heart valve leaflet (e.g., the left heart valve leaflet 20a, the right heart valve leaflet 20b). In some embodiments, positioning the ablative cutting element 122 against the heart valve leaflet (e.g., the left heart valve leaflet 20a, the right heart valve leaflet 20b) may comprise positioning the ablative cutting element 122 in contact with the nadir (e.g., refs. 28, 30) of the heart valve leaflet (e.g., the left heart valve leaflet 20a, the right heart valve leaflet 20b). In some embodiments, positioning the ablative cutting element 122 in contact with the nadir (e.g., refs. 28, 30) of the heart valve leaflet (e.g., the left heart valve leaflet 20a, the right heart valve leaflet 20b) may comprise shifting the expandable member 150 from the collapsed configuration toward and/or to the expanded configuration.

After advancing the expandable member 150 into the aortic valve 10 and/or within the plurality of heart valve leaflets 20 in the collapsed configuration, and/or after positioning the ablative cutting element 122 against the heart valve leaflet (e.g., the left heart valve leaflet 20a, the right heart valve leaflet 20b) and/or in contact with the nadir (e.g., refs. 28, 30) of the heart valve leaflet (e.g., the left heart valve leaflet 20a, the right heart valve leaflet 20b), the method of lacerating heart valve leaflets may comprise lacerating the heart valve leaflet (e.g., the left heart valve leaflet 20a, the right heart valve leaflet 20b) to the free edge (ref. 21) of the heart valve leaflet (e.g., the left heart valve leaflet 20a, the right heart valve leaflet 20b). In at least some embodiments, lacerating the heart valve leaflet (e.g., the left heart valve leaflet 20a, the right heart valve leaflet 20b) may comprise shifting the expandable member 150 from the collapsed configuration toward and/or to the expanded configuration, as seen in FIG. 11.

In some embodiments, shifting the expandable member 150 from the collapsed configuration toward and/or to the expanded configuration may comprise mechanically shifting the expandable member 150 from the collapsed configuration toward and/or to the expanded configuration. In some embodiments, the expandable member 150 may be configured to self-expand toward and/or to the expanded configuration. In some embodiments, shifting the expandable member 150 from the collapsed configuration toward and/or to the expanded configuration may comprise supplying an inflation fluid through the working lumen 134 and/or the inflation lumen into the expandable member 150. Other configurations are also contemplated.

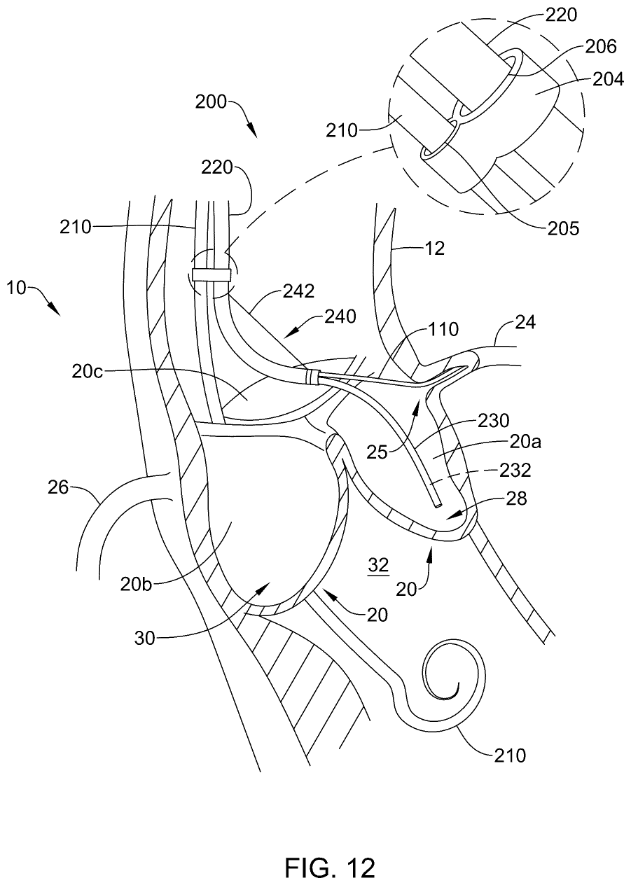

FIG. 12 illustrates selected aspects of a system 200 for lacerating heart valve leaflets. In some embodiments, the system 200 may comprise a first guidewire 210 configured for insertion into the left ventricle 32 of a heart. In some embodiments, the first guidewire 210 may be a guidewire compatible with and/or used during transcatheter aortic valve replacement (TAVR) and/or transcatheter aortic valve implantation (TAVI) procedures. One suitable example of a commercially available guidewire that may be used as the first guidewire 210 is the Safari2™ Guidewire from Boston Scientific. Other examples, including family members thereof, may also be considered. Some suitable but non-limiting materials for the first guidewire 210, etc., including but not limited to polymeric materials, metallic materials, and/or composite materials, are described below.

In some embodiments, a method of lacerating heart valve leaflets may comprise advancing the first guidewire 210 through the aortic valve 10 and/or the plurality of heart valve leaflets 20 into the left ventricle 32 of a patient's heart. In some embodiments, the method of lacerating heart valve leaflets may comprise positioning a distal end of the first guidewire 210 upstream of the aortic valve 10 and/or the plurality of heart valve leaflets 20. In some embodiments, the method of lacerating heart valve leaflets may comprise positioning the distal end of the first guidewire 210 within the left ventricle 32 of the patient's heart. In some embodiments, the method of lacerating heart valve leaflets may comprise positioning the first guidewire 210 between two heart valve leaflets generally opposite a heart valve leaflet (e.g., the left heart valve leaflet 20a, the right heart valve leaflet 20b) being lacerated. For example, the first guidewire 210 may be positioned between the right heart valve leaflet 20b and the non-coronary heart valve leaflet 20c when lacerating the left heart valve leaflet 20a, or between the left heart valve leaflet 20a and the non-coronary heart valve leaflet 20c when lacerating the right heart valve leaflet 20b. In some embodiments, the method of lacerating heart valve leaflets may comprise positioning the first guidewire 210 against a wall of the ascending aorta 12 generally opposite a coronary artery (e.g., the left coronary artery 24, the right coronary artery 26) associated with a heart valve leaflet (e.g., the left heart valve leaflet 20a, the right heart valve leaflet 20b) being lacerated.

In some embodiments, the system 200 may comprise a guide catheter 220 configured to translate along the first guidewire 210 to a position adjacent a heart valve leaflet (e.g., the left heart valve leaflet 20a, the right heart valve leaflet 20b) disposed proximate a coronary artery (e.g., the left coronary artery 24, the right coronary artery 26) associated with the heart valve leaflet (e.g., the left heart valve leaflet 20a, the right heart valve leaflet 20b). In some embodiments, the system 200 may comprise a coronary guidewire 110 having a distal end configured for insertion into the coronary artery (e.g., the left coronary artery 24, the right coronary artery 26) associated with the heart valve leaflet (e.g., the left heart valve leaflet 20a, the right heart valve leaflet 20b).

In some embodiments, the method of lacerating heart valve leaflets may comprise advancing and/or translating the guide catheter 220 along the first guidewire 210 to the position adjacent the heart valve leaflet (e.g., the left heart valve leaflet 20a, the right heart valve leaflet 20b) disposed proximate the coronary artery (e.g., the left coronary artery 24, the right coronary artery 26) associated with the heart valve leaflet (e.g., the left heart valve leaflet 20a, the right heart valve leaflet 20b).

In some embodiments, the system 200 may comprise a laceration device 230 slidably disposed within the guide catheter 220. In some embodiments, the laceration device 230 may comprise a tubular sheath. In some embodiments, the laceration device 230 may comprise an ablative cutting element 232 (not visible in FIG. 12). In some embodiments, the ablative cutting element 232 may be a conductive wire (e.g., a guidewire etc.) slidably disposed within the laceration device 230 and/or the tubular sheath. In some embodiments, the ablative cutting element 232 may be configured to be electrically coupled to a controller and/or an energy source, such as but not limited to a radiofrequency (RF) energy source, an RF generator, etc. Other configurations are also contemplated.

In some embodiments, the guide catheter 220 may be slidably coupled to the first guidewire 210 such that the guide catheter 220 is offset laterally from the first guidewire 210. In some embodiments, the guide catheter 220 may be slidably coupled to the first guidewire 210 such that the guide catheter 220 is disposed non-concentric with the first guidewire 210. In some embodiments, the guide catheter 220 may be slidably coupled to the first guidewire 210 such that the guide catheter 220 is disposed eccentrically with respect to the first guidewire 210. In some embodiments, the guide catheter 220 may be slidably coupled to the first guidewire 210 such that the guide catheter 220 is disposed alongside of the first guidewire 210. In some embodiments, the guide catheter 220 may be slidably coupled to the first guidewire 210 such that the first guidewire 210 is disposed outside of and/or exterior to the guide catheter 220. In some embodiments, the guide catheter 220 may be slidably coupled to the first guidewire 210 such that the first guidewire 210 is disposed completely outside of and/or completely exterior to the guide catheter 220.

In some embodiments, the guide catheter 220 may be slidably coupled to the first guidewire 210 by a sled 204. In some embodiments, the sled 204 may comprise a first lumen 205 and a second lumen 206 disposed side-by-side and/or laterally offset from each other. In some embodiments, the first lumen 205 and the second lumen 206 may be oriented parallel to each other. In some embodiments, the first lumen 205 may be sized and configured to slidably receive the first guidewire 210. In some embodiments, the second lumen 206 may be sized and configured to receive the guide catheter 220. In some embodiments, the sled 204 may be non-slidably coupled to the guide catheter 220. In some embodiments, the sled 204 may be fixedly attached to the guide catheter 220. In some embodiments, the second lumen 206 may be sized and configured to slidably receive the guide catheter 220. In some alternative embodiments, the guide catheter 220 may be slidably coupled to the first guidewire 210 by a plurality of sleds. Other configurations are also contemplated.

In some embodiments, the first guidewire 210 and/or the guide catheter 220 may optionally be slidably disposed within and/or may optionally be advanced toward the aortic valve 10 within a delivery sheath (not shown in FIG. 12). In some embodiments, the delivery sheath may slidably couple the guide catheter 220 to the first guidewire 210. In some embodiments, the system 200 may comprise the delivery sheath or the sled 204 (or the plurality of sleds). In some embodiments, the system 200 may comprise the delivery sheath and the sled 204. In some embodiments, the system 200 may comprise the delivery sheath and the plurality of sleds.

In some embodiments, the guide catheter 220 may comprise a guidewire lumen and a working lumen separate from the guidewire lumen. In some embodiments, the guide catheter 220 may be configured similarly to the device catheter 130 of FIG. 8. In at least some embodiments, the guidewire lumen and the working lumen may be completely separated and/or independent from each other (e.g., the guidewire lumen and the working lumen do not coexist within a common lumen). Other configurations are also contemplated. For example, in some alternative embodiments, a portion of the guidewire lumen and a portion of the working lumen may share and/or coexist within a common lumen or a portion of a lumen. However, such a configuration may not be preferred due to the risk of entanglement of the coronary guidewire 110 with the laceration device 230, which may increase operational forces, or cause other complications. In some embodiments, the coronary guidewire 110 may be sized and configured to extend through the guidewire lumen. In some embodiments, the laceration device 230, the ablative cutting element 232, and/or the conductive wire may be sized and configured to extend through the working lumen.

In some embodiments, the guide catheter 220 may comprise a steering element 240 configured to direct the coronary guidewire 110 toward the coronary artery (e.g., the left coronary artery 24 and/or the right coronary artery 26). In some embodiments, the steering element 240 may be a pull wire 242 configured to deflect a distal end of the guide catheter 220 toward the coronary artery (e.g., the left coronary artery 24 and/or the right coronary artery 26). In some embodiments, the pull wire 242 may extend within the guide catheter 220. In some embodiments, the pull wire 242 may extend alongside the guide catheter 220. In some embodiments, a first portion of the pull wire 242 may extend within the guide catheter 220 and a second portion of the pull wire 242 may extend alongside the guide catheter 220 proximate the distal end. Other configurations are also contemplated.

In some embodiments, the method of lacerating heart valve leaflets (e.g., the left heart valve leaflet 20a and/or the right heart valve leaflet 20b) may comprise advancing the coronary guidewire 110 from the guidewire lumen toward and/or into the coronary artery (e.g., the left coronary artery 24 and/or the right coronary artery 26). In some embodiments, the method of lacerating heart valve leaflets (e.g., the left heart valve leaflet 20a and/or the right heart valve leaflet 20b) may comprise anchoring the coronary guidewire 110 within the coronary artery (e.g., the left coronary artery 24 and/or the right coronary artery 26), as discussed herein.

In some embodiments, the laceration device 230 may be configured to cooperate with the coronary guidewire 110 to align the laceration device 230 and/or the ablative cutting element 232 with the coronary guidewire 110, the coronary artery (e.g., the left coronary artery 24, the right coronary artery 26) associated with a heart valve leaflet (e.g., the left heart valve leaflet 20a, the right heart valve leaflet 20b) being lacerated, and/or the nadir (e.g., the nadir 28, the nadir 30) of the heart valve leaflet (e.g., the left heart valve leaflet 20a, the right heart valve leaflet 20b) being lacerated. In some embodiments, the laceration device 230 may be configured to cooperate with the coronary guidewire 110 to align the laceration device 230 and/or the ablative cutting element 232 with the nadir (e.g., the nadir 28, the nadir 30) of the heart valve leaflet (e.g., the left heart valve leaflet 20a, the right heart valve leaflet 20b). Other configurations are also contemplated. In some embodiments, the laceration device 230 may be aligned with the coronary guidewire 110 under fluoroscopy (or another suitable visualization means) using one or more views such that a distal end of the laceration device 230 may be directed toward and/or to the nadir (e.g., the nadir 28, the nadir 30) of the heart valve leaflet (e.g., the left heart valve leaflet 20a, the right heart valve leaflet 20b). In some embodiments, the laceration device 230 may comprise one or more radiopaque markers configured to facilitate aligning the laceration device 230 and/or the ablative cutting element 232 with the coronary guidewire 110 and/or the nadir (e.g., the nadir 28, the nadir 30) of the heart valve leaflet (e.g., the left heart valve leaflet 20a, the right heart valve leaflet 20b).

In some embodiments, the laceration device 230 may be configured to position a distal end of the laceration device 230 adjacent to the nadir (e.g., the nadir 28, the nadir 30) of the heart valve leaflet (e.g., the left heart valve leaflet 20a, the right heart valve leaflet 20b). In some embodiments, the method of lacerating heart valve leaflets may comprise positioning the ablative cutting element 232 in contact with the nadir (e.g., the nadir 28, the nadir 30) of the heart valve leaflet (e.g., the left heart valve leaflet 20a, the right heart valve leaflet 20b). In some embodiments, positioning the ablative cutting element 232 in contact with the nadir (e.g., the nadir 28, the nadir 30) of the heart valve leaflet (e.g., the left heart valve leaflet 20a, the right heart valve leaflet 20b) comprises orienting the guide catheter 220, with the coronary guidewire 110 disposed within the coronary artery (e.g., the left coronary artery 24 and/or the right coronary artery 26), such that the working lumen opens toward the nadir (e.g., the nadir 28, the nadir 30) of the heart valve leaflet (e.g., the left heart valve leaflet 20a, the right heart valve leaflet 20b).

In some embodiments, positioning the ablative cutting element 232 in contact with the nadir (e.g., the nadir 28, the nadir 30) of the heart valve leaflet (e.g., the left heart valve leaflet 20a, the right heart valve leaflet 20b) comprises advancing the ablative cutting element 232 out of the working lumen into contact with the nadir (e.g., the nadir 28, the nadir 30) of the heart valve leaflet (e.g., the left heart valve leaflet 20a, the right heart valve leaflet 20b). In at least some embodiments, positioning the ablative cutting element 232 in contact with the nadir (e.g., the nadir 28, the nadir 30) of the heart valve leaflet (e.g., the left heart valve leaflet 20a, the right heart valve leaflet 20b) comprises extending the ablative cutting element 232 through the nadir (e.g., the nadir 28, the nadir 30) of the heart valve leaflet (e.g., the left heart valve leaflet 20a, the right heart valve leaflet 20b), similar to the configuration shown in FIG. 5. As discussed herein, the ablative cutting element 232 may be and/or may comprise a conductive wire. In some embodiments, positioning the ablative cutting element 232 in contact with the nadir (e.g., the nadir 28, the nadir 30) of the heart valve leaflet (e.g., the left heart valve leaflet 20a, the right heart valve leaflet 20b) comprises extending the conductive wire through the nadir (e.g., the nadir 28, the nadir 30) of the heart valve leaflet (e.g., the left heart valve leaflet 20a, the right heart valve leaflet 20b). In some embodiments, the ablative cutting element 232 and/or the conductive wire may be configured to puncture the nadir (e.g., the nadir 28, the nadir 30) of the heart valve leaflet (e.g., the left heart valve leaflet 20a, the right heart valve leaflet 20b).

In some embodiments, the system 200 for lacerating heart valve leaflets may comprise a second device catheter 140 (not shown in FIG. 12). The second device catheter 140 may be configured to be advanced percutaneously through the plurality of heart valve leaflets 20 such that a distal end of the second device catheter 140 is disposed upstream of and/or on an upstream side of the plurality of heart valve leaflets 20, as seen in FIG. 5 for example. Accordingly, the method of lacerating heart valve leaflets may comprise advancing the second device catheter 140 through the plurality of heart valve leaflets 20 and/or positioning a distal end of the second device catheter 140 upstream of and/or on an upstream side of the plurality of heart valve leaflets 20.

The second device catheter 140 may comprise a snare 142 (not shown in FIG. 12). In some embodiments, the snare 142 may extend from the second device catheter 140. In some embodiments, the snare 142 may be slidably disposed within the second device catheter 140 and be configured to be extended from the second device catheter 140. In some embodiments, the snare 142 may be configured to capture and/or grasp the ablative cutting element 232 and/or the conductive wire. In some embodiments, the snare 142 may comprise a closed loop. In some embodiments, the snare 142 may comprise a cinch loop. In some embodiments, the snare 142 may comprise an actuatable grasping element, such as a clip (e.g., an alligator clip). In some embodiments, capturing and/or grasping the ablative cutting element 122 and/or the conductive wire may comprise closing the snare 142 around and/or onto the ablative cutting element 122 and/or the conductive wire. The method of lacerating heart valve leaflets may comprise capturing and/or grasping the ablative cutting element 232 and/or the conductive wire with the snare 142.

In at least some embodiments, the snare 142 may be configured to pull a distal end portion of the ablative cutting element 232 and/or the conductive wire into the second device catheter 140. In some embodiments, the method of lacerating heart valve leaflets may comprise, after capturing and/or grasping the ablative cutting element 232 and/or the conductive wire with the snare 142, pulling the distal end portion of the ablative cutting element 232 and/or the conductive wire into the second device catheter 140.

In some embodiments, the snare 142 may be formed from a metallic material (e.g., nickel-titanium alloy, stainless steel and/or stainless-steel alloy, etc.), a polymeric material (e.g., nylon, aramid, PET, graphene, etc.), or a composite material. In some embodiments, the snare 142 may be formed from a high-tensile fiber or filament. In some embodiments, non-metallic materials may be doped with a radiopaque media (e.g., iodine, tungsten, platinum-iridium alloy, etc.). Other configurations are also contemplated. Some suitable but non-limiting materials for the snare 142, etc., including but not limited to polymeric materials, metallic materials, and/or composite materials, are described below.

In some embodiments, the method of lacerating heart valve leaflets may comprise withdrawing and/or removing the coronary guidewire 110 from the coronary artery (e.g., the left coronary artery 24 and/or the right coronary artery 26). In some embodiments, withdrawing and/or removing the coronary guidewire 110 from the coronary artery (e.g., the left coronary artery 24 and/or the right coronary artery 26) may comprise withdrawing and/or removing the coronary guidewire 110 from the patient and/or the patient's vasculature. In some embodiments, withdrawing and/or removing the coronary guidewire 110 from the coronary artery (e.g., the left coronary artery 24 and/or the right coronary artery 26) may comprise withdrawing the coronary guidewire 110 into the delivery sheath. In some embodiments, withdrawing and/or removing the coronary guidewire 110 from the coronary artery (e.g., the left coronary artery 24 and/or the right coronary artery 26) may comprise withdrawing the coronary guidewire 110 into the guide catheter 220. Other configurations are also contemplated.

In some embodiments, the method of lacerating heart valve leaflets may comprise lacerating the heart valve leaflet (e.g., the left heart valve leaflet 20a, the right heart valve leaflet 20b) from the nadir (e.g., refs. 28, 30) of the heart valve leaflet (e.g., the left heart valve leaflet 20a, the right heart valve leaflet 20b) to a free edge (e.g., ref. 21) of the heart valve leaflet (e.g., the left heart valve leaflet 20a, the right heart valve leaflet 20b), as seen in FIG. 7. In some embodiments, lacerating the heart valve leaflet (e.g., the left heart valve leaflet 20a, the right heart valve leaflet 20b) may comprise translating the laceration device 230 and/or the guide catheter 220 proximally along with and/or concurrently with the second device catheter 140 to lacerate the heart valve leaflet (e.g., the left heart valve leaflet 20a, the right heart valve leaflet 20b) axially and/or in a downstream direction aligned with the ostium (e.g., the left coronary ostium 25, the right coronary ostium 27) of the coronary artery (e.g., the left coronary artery 24, the right coronary artery 26) such that the heart valve leaflet (e.g., the left heart valve leaflet 20a, the right heart valve leaflet 20b) is cut into two adjacent portions. In some embodiments, lacerating the heart valve leaflet (e.g., the left heart valve leaflet 20a, the right heart valve leaflet 20b) may comprise translating the laceration device 230 and/or the guide catheter 220 proximally along the first guidewire 210 and/or through the sled 204. Other configurations are also contemplated.

In at least some embodiments, prior to lacerating the heart valve leaflet (e.g., the left heart valve leaflet 20a, the right heart valve leaflet 20b), the method may comprise energizing the ablative cutting element 232 and/or the conductive wire. In some embodiments, energizing the ablative cutting element 232 and/or the conductive wire may comprise applying RF energy to the ablative cutting element 232 and/or the conductive wire. Other configurations are also contemplated.

Some suitable but non-limiting materials for the delivery sheath, the sled 204, the guide catheter 220, the laceration device 230, the pull wire 242, etc., including but not limited to polymeric materials, metallic materials, and/or composite materials, are described below.

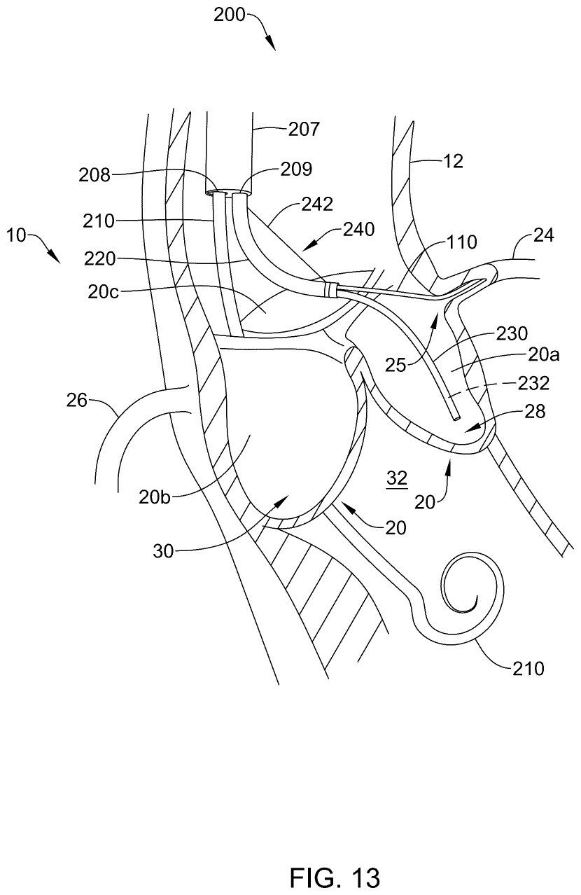

In some embodiments, the system 200 may comprise a tubular member 207 instead of the sled 204 (e.g., FIG. 12), as seen in FIG. 13. The tubular member 207 may comprise a plurality of lumens extending therein. In some embodiments, the tubular member 207 may comprise two or more nested tubular members (e.g., one or more tubular members may be disposed within an outer tubular member). In some embodiments, the tubular member 207 may comprise a first lumen 208 and a second lumen 209. In some embodiments, the first lumen 208 and the second lumen 209 may extend side-by-side and/or laterally offset from each other within the tubular member 207. In some embodiments, the first lumen 208 and the second lumen 209 may be oriented parallel to each other. In some embodiments, the first lumen 208 may be sized and configured to slidably receive the first guidewire 210. In some embodiments, the second lumen 209 may be sized and configured to slidably receive the guide catheter 220. Other configurations are also contemplated.

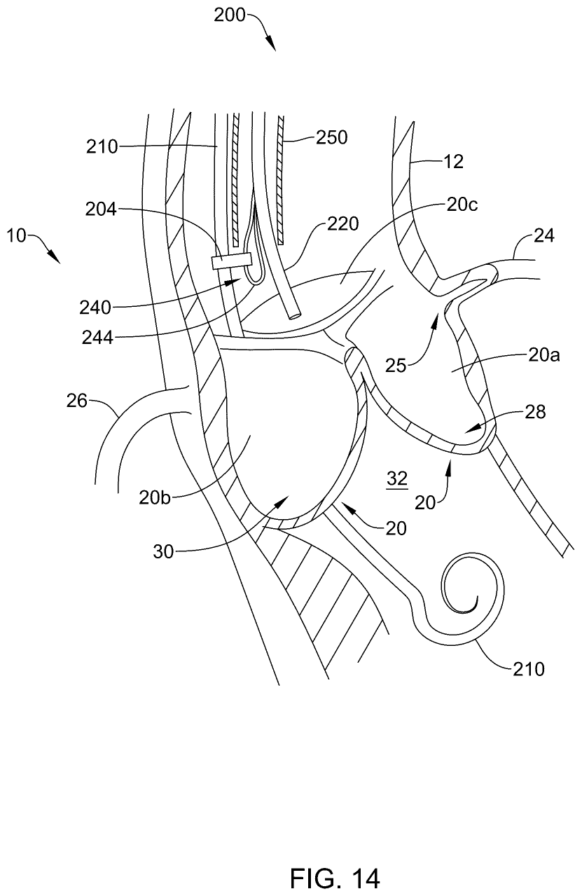

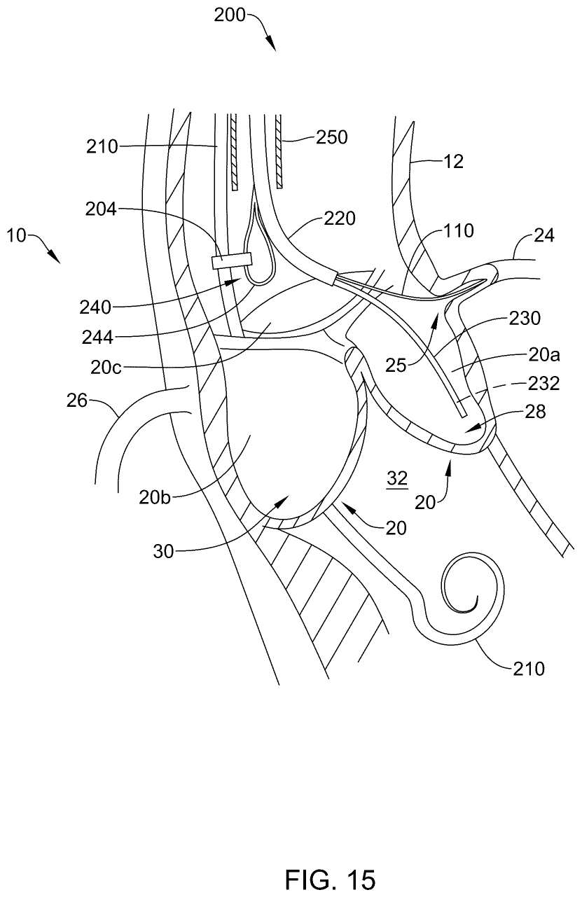

FIGS. 14-15 illustrate selected aspects of an alternative configuration of the system 200 for lacerating heart valve leaflets. In some embodiments, the steering element 240 comprises an expandable frame 244 (instead of the pull wire 242) configured to shift from a delivery configuration (e.g., FIG. 14) to a deployed configuration (e.g., FIG. 15) to deflect the distal end of the guide catheter 220 toward the coronary artery (e.g., the left coronary artery 24, the right coronary artery 26) in the deployed configuration. In at least some embodiments, the expandable frame 244 may be self-expanding and/or may be self-biased toward the deployed configuration.

In some embodiments, the expandable frame 244 may be fixedly attached to the guide catheter 220. In some embodiments, the expandable frame 244 may be fixedly attached to a branch of the guide catheter 220 such that the expandable frame 244 extends alongside a distal portion of the guide catheter 220 near the distal end of the guide catheter 220. In some embodiments, the expandable frame 244 may comprise and/or may form a closed loop. In some embodiments, the expandable frame 244 and/or the closed loop may be slidably coupled to the first guidewire 210. In some embodiments, the expandable frame 244 and/or the closed loop may be slidably coupled to the first guidewire 210 by the sled 204. Other configurations are also contemplated. In some embodiments, the sled 204 may be configured to retain at least a portion of the expandable frame 244 in close proximity to the first guidewire 210.

In some embodiments, the system 200 may comprise a delivery catheter 250 configured to constrain the expandable frame 244 in the delivery configuration during translation of the guide catheter 220 along the first guidewire 210, as seen in FIG. 14. The delivery catheter 250 is shown in cross-section to illustrate elements/structures disposed therein. Proximal movement of the delivery catheter 250 relative to the guide catheter 220 and/or the expandable frame 244 may permit the expandable frame 244 to shift toward and/or to the deployed configuration, as seen in FIG. 15. Other configurations are also contemplated.

In some embodiments, the expandable frame 244 may be formed from a shape memory material. In one non-limiting example, the expandable frame 244 may be formed from nickel-titanium alloy (e.g., nitinol). Some suitable but non-limiting materials for the expandable frame 244, the delivery catheter 250, etc., including but not limited to polymeric materials, metallic materials, and/or composite materials, are described below.

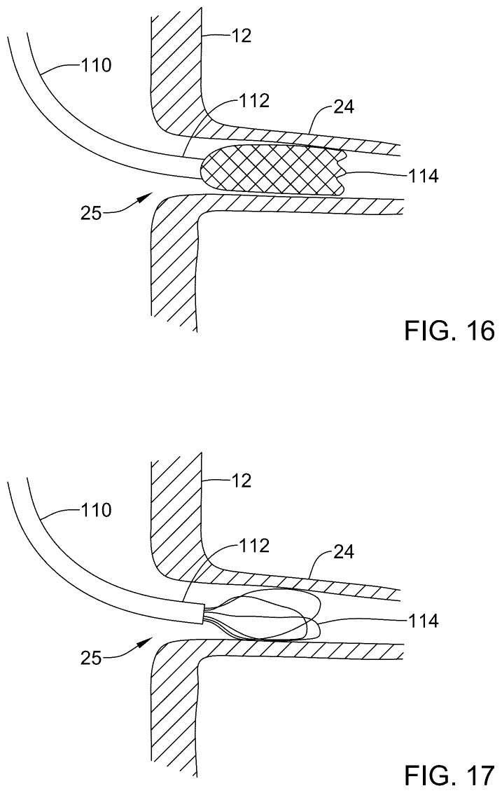

FIGS. 16-21 illustrate selected aspects of some embodiments of the coronary guidewire 110. As discussed herein, in some embodiments, the coronary guidewire 110 may have a distal end 112 configured for insertion into a coronary artery (e.g., the left coronary artery 24 and/or the right coronary artery 26). In some embodiments, the distal end 112 of the coronary guidewire 110 may comprise an expandable structure 114 configured to anchor the coronary guidewire 110 and/or the distal end 112 of the coronary guidewire 110 within the coronary artery (e.g., the left coronary artery 24 and/or the right coronary artery 26) in an expanded configuration. In some embodiments, the expandable structure 114 may be configured to engage with the coronary artery (e.g., the left coronary artery 24 and/or the right coronary artery 26) in the expanded configuration to provide stability and/or light anchoring. In at least some embodiments, the expandable structure 114 may be configured to avoid dilating and/or expanding the coronary artery (e.g., the left coronary artery 24 and/or the right coronary artery 26) in the expanded configuration. In some embodiments, the expandable structure 114 may comprise and/or may be enhanced with radiopaque material(s) and/or radiopaque elements for visualization and/or confirmation of placement, under fluoroscopy for example. In some embodiments, the expandable structure 114 may comprise a coating disposed thereon. In some embodiments, the coating may be configured to reduce abrasion of the coronary artery (e.g., the left coronary artery 24 and/or the right coronary artery 26). In some embodiments, the coating may be formed from a hydrophilic material.

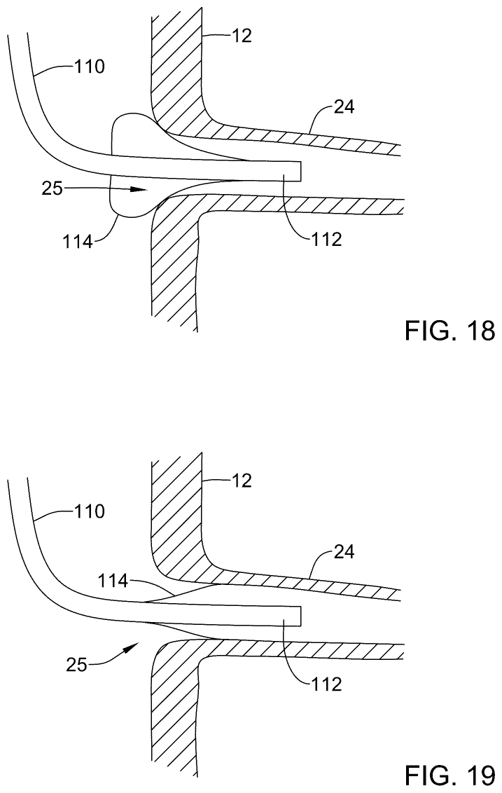

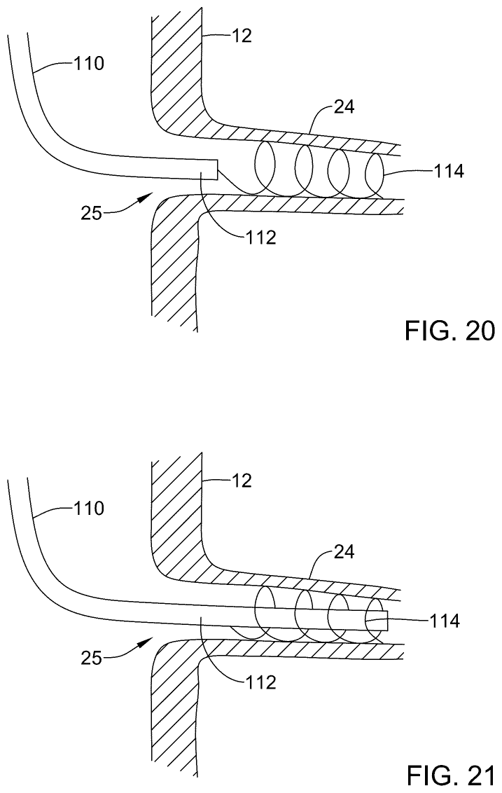

In some embodiments, the expandable structure 114 may comprise a stent or a stent-like structure extending from the coronary guidewire 110 in the expanded configuration, as seen in FIG. 16. In some embodiments, the expandable structure 114 may comprise an expandable wire frame extending from the coronary guidewire 110 in the expanded configuration, as seen in FIG. 17. In some embodiments, the expandable structure 114 may comprise a teardrop frame extending from the coronary guidewire 110 in the expanded configuration, as seen in FIG. 18. In some embodiments, the expandable structure 114 may comprise a plurality of wires extending from the coronary guidewire 110 in a distal direction and in a radially outward direction to free ends spaced apart from the coronary guidewire 110 in the expanded configuration, as seen in FIG. 19. In some embodiments, the expandable structure 114 may comprise a helical coil extending distally from the coronary guidewire 110 in the expanded configuration, as seen in FIG. 20. In some embodiments, the expandable structure 114 may comprise a helical coil extending around the coronary guidewire 110 in the expanded configuration, as seen in FIG. 21. In at least some embodiments, the expandable structure 114 may be configured to permit blood or fluid to flow through the coronary artery (e.g., the left coronary artery 24 and/or the right coronary artery 26) past the expandable structure 114 in the expanded configuration. In some embodiments, the expandable structure 114 may be configured to permit a user to apply positive pressure to the coronary guidewire 110 to maintain position of the coronary guidewire 110 within the coronary artery (e.g., the left coronary artery 24 and/or the right coronary artery 26).

In some alternative embodiments, the expandable structure 114 may comprise an inflatable balloon. However, in such embodiments, the expandable structure 114 and/or the inflatable balloon should include a means of perfusion (e.g., structure permitting blood or fluid to flow) through the coronary artery (e.g., the left coronary artery 24 and/or the right coronary artery 26) past the expandable structure 114 and/or the inflatable balloon in the expanded configuration. In some embodiments, the inflatable balloon may be inflated with radiopaque media to confirm placement and/or visibility of location within the coronary artery (e.g., the left coronary artery 24 and/or the right coronary artery 26). In some embodiments, the inflatable balloon may be inflated with radiopaque media to permit visualization of dilatation of the inflatable balloon and/or the coronary artery (e.g., the left coronary artery 24 and/or the right coronary artery 26). In at least some embodiments, the inflatable balloon may be adapted and configured to provide anchoring and/or securement within the coronary artery (e.g., the left coronary artery 24 and/or the right coronary artery 26) and/or to avoid dilating and/or expanding the coronary artery (e.g., the left coronary artery 24 and/or the right coronary artery 26) in the expanded configuration. In some embodiments, the inflatable balloon may be formed from a compliant polymeric material (e.g., silicone, etc.).

Some suitable but non-limiting materials for the expandable structure 114, the inflatable balloon, the coating, etc., including but not limited to polymeric materials, metallic materials, and/or composite materials, are described below.

In some embodiments, devices and systems disclosed herein may be used to treat and/or lacerate more than one heart valve leaflet during a procedure. In some embodiments, devices and systems disclosed herein may be used to treat and/or lacerate only one heart valve leaflet during a procedure, such that multiple devices and/or systems may be used to treat more than one heart valve leaflet during the procedure. In some embodiments, methods and/or steps thereof disclosed herein may be repeated for each heart valve leaflet being treated and/or lacerated during a procedure.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the coronary guidewire, the laceration device, the device catheter, the guide catheter, the delivery catheter, the delivery sheath, the expandable member, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, acrylonitrile butadiene styrene (ABS), epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; stainless-steel alloys, mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such HASTELLOY® C-22®, UNS: N10276 such as HASTELLOY® C276®, other as HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-NR and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment in other embodiments. The scope of the disclosure is, of course, defined in the language in which the appended claims are expressed.

What is claimed is:

1. A system for lacerating heart valve leaflets, comprising:
a coronary guidewire having a distal end configured for insertion into a coronary artery; and
a laceration device configured to translate along the coronary guidewire to a position adjacent a heart valve leaflet disposed proximate the coronary artery, wherein the laceration device comprises an ablative cutting element;
wherein the laceration device cooperates with the coronary guidewire to align the ablative cutting element with a nadir of the heart valve leaflet.

2. The system of claim 1, wherein the distal end of the coronary guidewire comprises an expandable structure configured to anchor the coronary guidewire within the coronary artery in an expanded configuration.

3. The system of claim 2, wherein the expandable structure is configured to permit blood to flow through the coronary artery past the expandable structure in the expanded configuration.

4. The system of claim 1, wherein the laceration device is slidably coupled to the coronary guidewire such that the laceration device is offset laterally from the coronary guidewire.

5. The system of claim 1, wherein the laceration device comprises an expandable member disposed at a distal end of the laceration device, wherein the ablative cutting element is disposed on the expandable member.

6. The system of claim 1, wherein the laceration device comprises a device catheter comprising a guidewire lumen and a working lumen separate from the guidewire lumen, wherein the coronary guidewire is configured to extend through the guidewire lumen and the ablative cutting element is configured to extend through the working lumen.

7. The system of claim 6, wherein the device catheter is bifurcated proximate its distal end such that the guidewire lumen and the working lumen diverge from each other.

8. The system of claim 7, wherein the device catheter is configured such that with the coronary guidewire disposed within the coronary artery, the working lumen extends toward the nadir of the heart valve leaflet.

9. A system for lacerating heart valve leaflets, comprising:
a first guidewire configured for insertion into a left ventricle of a heart;
a guide catheter configured to translate along the first guidewire to a position adjacent a heart valve leaflet disposed proximate the coronary artery;
a laceration device slidably disposed within the guide catheter, the laceration device comprising an ablative cutting element; and
a coronary guidewire having a distal end configured for insertion into a coronary artery;
wherein the guide catheter comprises a steering element configured to direct the coronary guidewire toward the coronary artery;
wherein the laceration device cooperates with the coronary guidewire to align the ablative cutting element with a nadir of the heart valve leaflet.

10. The system of claim 9, wherein the steering element is a pull wire configured to deflect a distal end of the guide catheter toward the coronary artery.

11. The system of claim 9, wherein the steering element is an expandable frame configured to shift from a delivery configuration to a deployed configuration to deflect a distal end of the guide catheter toward the coronary artery.

12. The system of claim 11, further comprising a delivery catheter configured to constrain the expandable frame in the delivery configuration during translation of the guide catheter along the first guidewire, wherein proximal movement of the delivery catheter relative to the guide catheter permits the expandable frame to shift to the deployed configuration.

13. The system of claim 11, wherein the expandable frame is fixedly attached to the guide catheter.

14. The system of claim 11, wherein the expandable frame is slidably coupled to the first guidewire.

15. A method of lacerating heart valve leaflets, comprising:
advancing a coronary guidewire into a coronary artery;
translating a laceration device along the coronary guidewire to a position adjacent a heart valve leaflet disposed proximate the coronary artery, wherein the laceration device comprises an ablative cutting element;
positioning the ablative cutting element in contact with a nadir of the heart valve leaflet; and
lacerating the heart valve leaflet from the nadir of the heart valve leaflet to a free edge of the heart valve leaflet.

16. The method of claim 15, further comprising:
before translating the laceration device along the coronary guidewire, anchoring the coronary guidewire within the coronary artery.

17. The method of claim 15, wherein positioning the ablative cutting element in contact with the nadir of the heart valve leaflet comprises shifting an expandable member from a collapsed configuration to an expanded configuration.

18. The method of claim 17, wherein the expandable member is disposed at a distal end of a device catheter and the coronary guidewire is disposed within a guidewire lumen of the device catheter, wherein the coronary guidewire exits the guidewire lumen proximal of the expandable member.

19. The method of claim 15, wherein the laceration device comprises a device catheter comprising a guidewire lumen and a working lumen separate from the guidewire lumen, wherein the coronary guidewire is configured to extend through the guidewire lumen and the ablative cutting element is configured to extend through the working lumen.

20. The method of claim 19, wherein positioning the ablative cutting element in contact with the nadir of the heart valve leaflet comprises:
orienting the device catheter, with the coronary guidewire disposed within the coronary artery, such that the working lumen opens toward the nadir of the heart valve leaflet; and
advancing the ablative cutting element out of the working lumen into contact with the nadir of the heart valve leaflet.
